(19) United States
(12) Patent Application Publication
(10) Pub. No.: US 2023/0241326 A1
(43) Pub. Date: Aug. 3, 2023

(54) ACTIVATION AND DETECTION SYSTEM FOR A SUPPLEMENTAL DEVICE ATTACHED TO A MEDICAMENT DELIVERY DEVICE

(71) Applicant: SHL MEDICAL AG, ZUG (CH)

(72) Inventor: Antonio Farieta, Baar (CH)

(21) Appl. No.: 17/923,315

(22) PCT Filed: Jun. 22, 2021
(86) PCT No.: PCT/EP2021/066992

§ 371 Date: Nov. 8, 2022

(30) Foreign Application Priority Data
Jul. 6, 2020 (EP) 20184146.7

(51) Int. Cl. A61M 5/32; A61M 5/315

(52) U.S. Cl. CPC A61M 5/3204; A61M 5/31571; A61M 2205/3306

(57) ABSTRACT

The present disclosure relates to an activation system for a supplemental device that is attached to an existing design of a medicament delivery device, where a movement applied on the protective cap or other covering of the medicament delivery device triggers the activation mechanism for re-establishing electrical contacts to close a circuit between a battery module and a power source, thus activating the battery module. The activation mechanism is configured to not intervene with the sterility of the dose delivery outlet. Thus, the supplemental device can be activated at any time before removal of the protective cap or other covering. The supplemental device can further have a transmitter for communicating information about the use of the medicament delivery device to an external device, such as a smart device. Memory storage elements can also be included having unique identification data. The present disclosure also relates to a medicament delivery device having the supplemental device attached.

### CROSS REFERENCE TO RELATED APPLICATIONS

The present application is a U.S. National Phase Application pursuant to 35 U.S.C. § 371 of International Application No. PCT/EP2021/066992 filed Jun. 22, 2021, which claims priority to European Patent Application No. 20184146.7 filed Jul. 6, 2020. The entire disclosure contents of these applications are herewith incorporated by reference into the present application.

### TECHNICAL FIELD

The present disclosure relates to an event detection mechanism and activation of a supplemental device that can be attached to a medicament delivery device, where the supplemental device can function as an information provider that is capable of transmitting unique information concerning the medicament delivery device. The supplemental device is activated by the removal of a covering from the medicament delivery device. The movement of a protective covering relative to the medical delivery device is detectable. The protective covering of the medicament delivery device may comprise an assembly of rigid and/or flexible needle shields and a needle shield remover. The supplemental device can monitor the use or the movement of the protective covering of the medicament delivery device and can communicate with external smart devices.

### BACKGROUND

Medicament delivery devices, especially those designed for medicament delivery by self-administration, have been on the market for a number of years. In order for the devices to be handled by non-professionals, they have to be easy to use and intuitive. Further, since many of the medicaments are vital or at least very important to the patient there is a desire from physicians and other professionals to obtain information that the patients medicate according to prescribed schemes. The desired information could include the type of medicament, delivery times, dates, dose size, and safety information such as sterility. Also of importance is whether the medicament or the device itself is counterfeit or has been tampered with. Methods and systems for detecting an open container while preserving the ability of tags and devices to communicate wirelessly are known. For example, U.S. Pat. No. 9,519,904 B2 discloses a method pertaining to radio frequency (RF and/or RFID) and near field communication (NFC) tags and devices with a mechanism for detecting an opened package or container, the continuity state of the package or container is determined to be opened when at least one of the protection line(s) is broken. Alternatively, or additionally, the continuity state of the package or container may be determined to be closed or sealed when one or more of the protection lines is not broken.

However, one drawback with the solution according to U.S. Pat. No. 9,519,904 B2 is that the power supply may provide the sensors and circuits with electricity in a continuous manner instead of being triggered by an action initiating the supply of power to the system. Accordingly, power supply i.e. battery storage may have a limited lifetime. Another drawback is that the activation occurs only after the protection line has been broken, in other words after the sterile barrier has been breached. An activation of the device prior to the breach of the sterile barrier is thus not available and transmission of information to an external device may not occur at this stage. However, there may be circumstances where information about the device prior to the breach of sterility are beneficial or needed.

Likewise, information that could be beneficial to the physician concerns whether the medicament has been taken using the correct procedure and following instructions for use; that the medicament has been maintained at the prescribed temperature before and during medicament delivery; that the right injection depth has been used and that the correct injection speed has been used, when the medicament delivery device is an injector.

Systems for obtaining information from a medicament delivery device are known. For example, WO 2004/084116 discloses a system for presenting and distributing medication information, where a medicament delivery device is arranged with communication mechanisms which will enable communication with an external device such as a cellular or a mobile phone or a personal digital assistant (PDA). A preferred communication standard is Bluetooth. The medicament delivery device is arranged with a number of sensors for monitoring and registering e.g. a dose delivery sequence. The idea is then to use the functionality of the external device, such as its display, its processor, its keyboard, etc. instead of providing the medicament delivery device with such features. The transfer of the functionality to the external device will reduce the cost of the medicament delivery device in comparison with medicament delivery devices provided with such functionality.

However, a drawback with the solution according to WO 2004/084116 is that a Bluetooth circuit, or another wireless communication systems such as ANT or ZigBee, is built into the housing of the medicament delivery device. The communication system with its battery to power the circuit requires a dedicated space in a medicament delivery device. Manipulation of existing device designs to accommodate an add-on battery module may trigger unforeseen regulatory problems. Another issue with known data collection devices is that the power source is directly connected to the electronic circuit at the time the battery module is manufactured and attached to the medicament delivery device. This can cause premature battery drain. Additionally, such systems require the user to perform a separate and specific step of activating the battery module, which may not always be performed successfully. Consequently, a breach of the sterility may not be detected when occurred after the battery is drained or was not successfully activated by the user.

It is thus not so easy to modify existing designs or to easily provide existing designs with added functionality that communication systems can provide. As such, there is a need to provide an activation system that is automatic and is performed prior to a breach of the sterility barrier, as part of the normal use of the medicament delivery device, providing an detection mechanism activated by the removal sequence of the protective unit before the sterility barrier is removed, thereby activating the supplemental device for recording and signalling the breach of the sterility barrier of the medical delivery device.

### SUMMARY

The aim of the present disclosure is to provide an event detection system, triggered by an integrated activation mechanism of the medicament delivery device prior to a breach of the sterility barrier, for a supplemental device, that is attachable to an existing medicament delivery device. The attachment does not require that the existing design of the medicament delivery device be modified or otherwise changed. A particularly preferred supplemental device is an information provider device that can be used on and with a number of different medicament delivery devices, and in particular configured for use on medicament delivery devices for self-administration of medicaments. Preferably the medicament delivery devices arranged with such information providers could be used with conventional external smart devices common on the market and used by the majority of patients that handle medicament delivery devices for self-administration.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the medicament delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

As used herein, the term “medicament” is meant to encompass any medicament-containing flowable medicine capable of being passed through a delivery means such as a cannula or hollow needle in a controlled manner, such as a liquid, solution, gel or fine suspension. Representative medicaments include pharmaceuticals such as peptides, proteins, and hormones, biologically derived or active agents, hormonal and gene-based agents, nutritional formulas and other substances in both solid (dispensed) or liquid form. In the description of the exemplary embodiments, reference will be made to the use of insulin. Correspondingly, the term “subcutaneous” infusion or injection is meant to encompass any method of transcutaneous delivery to a subject.

In the present disclosure the term “module” is meant to encompass a self-contained unit or item, such as an electronic component or an assembly of electronic components and associated wiring, which itself performs a defined task and can be linked with other such units to form a larger system.

According to a main aspect of the present disclosure, the supplemental device is powered by a battery that is initially in a disconnected state such that no power is supplied to or from the supplemental device. The activation mechanism of the supplemental device is preferably linked to the normal use of the medicament delivery device. Upon movement of the protective cover unit, an activation element is triggered by the removal sequence of the protective cap performed by a user. The removal sequence activating the supplemental device is preliminary to the axial movement of the needle shield away from the proximal end of the needle. The removal sequence of the cap required for activating the supplemental device is tolerated and the sterile barrier is not moved or breached by said movement. In other words, the activation mechanism does not destroy the condition of sterility of the delivery outlet.

According to a main aspect, the activation mechanism reconnects the battery to power the supplemental device prior to the breach of the sterility barrier. The battery is maintained in the isolated or disconnected state by a switch or insulating material, generally referred to hereafter as an activation member. The removal of the activation member from between the electrical contacts is analogous to the closing of a conventional line switch. Holding the normally closed switch open can be achieved by a mechanical, spatial or functional configuration applied to the switch. Likewise, removal of the insulating material will cause electrical contacts to close or otherwise come together to form an electrical connection such that electricity from the battery will flow through and between the contacts, thus closing or energizing the electrical circuit connected to the other electrical components.

Preferably, the supplemental device can be attached to the medicament delivery device prior to use the of the device, for example, during manufacturing or assembly, in a non-energized state, where a battery is temporarily electrically isolated from electrical connection with the other components of the supplemental device.

The activation member is configured to be in contact with the housing or the cap of the medicament delivery device when the supplemental device is mounted on the medicament delivery device. In the case of the mechanical switch configuration as an activation member, the switch is arranged to be slidably in contact with the surface of the proximal end of the medical delivery device. When the supplemental device is mounted on an existing injection device, such as an auto-injector, that has a protective cap that surrounds and encloses a separate needle cover or shield, and a pulling or twisting motion is applied to the cap by a user, the cap will move towards the proximal end of the device.

The portion of axial movement of the cap towards the proximal end of the device creates a gap between the housing and the cap. The cap abuts the proximal end of the housing such that the switch is in mechanical contact with the housing of the medicament delivery device. Upon movement of the cap toward the proximal end of the delivery device, the switch, triggered by the movement of the cap, slides along the housing of the medicament delivery device. When the switch reaches the terminal section of the proximal end of the housing, the switch can slide vertically into the space created by the distancing of the cap from the housing of the medical delivery device. This vertical movement with respect to the longitudinal axis of the medicament delivery device triggers the switch back to its default position and closes the circuit, to form an electrical connection such that electricity from the battery will flow through and between the contacts, thus closing or energizing the electrical circuit connected to the other electrical components.

Alternatively a circumferential or axial movement or a combination of an axial and circumferential movement of the cap involves the removal of the activation member, e.g., an insulating sheet or strip, from electrical contacts that are directly in contact with a battery that is part of an electrical circuit of the supplemental device that can contain a switch that is operatively connected to the battery and to a contact pad, where a part of the switch is directly connected to the cap.

The physical removal sequence or movement of the cap is preferably accomplished as part of a required step of using the medicament delivery device. For example, in the case of a medicament delivery device, the removal of a protective cap or other covering of a dose delivery outlet member is usually required prior to the use of the medicament delivery device. Incorporation of the procedure of the activation member with the removal sequence of the cap is a preferred mechanism because this eliminates the need for the user of the device to perform a separate step or procedure, and the supplemental device can be activated before the needle shield is moved. Especially in the case when the dose delivery outlet is an injection needle having its own separate flexible needle shield that is axially fixed to the protective cap, the movement of the cap away from the device housing therefore results in axial movement of the needle shield away from the proximal end of the needle which destroys the condition of sterility.

The coupling between the protective cap and the needle shield remover may be configured to allow a predefined axial displacement of the cap with respect to the needle shield remover, and this axial movement does not engage the needle shield remover to remove the needle shield. One example comprises an additional spacing between the gripping members of the cap and the needle shield remover. Stated differently the cap may slide with an axial and/or rotational movement along the needle shield remover, defining an axial displacement, before the gripping members of the needle shield and the gripping members of the cap can engage with each other. Upon coupling of the gripping members, any further axial and/or rotational movement of the cap translates into a movement of the needle shield remover and the attached rigid or flexible needle shield.

One embodiment may be configured to provide an additional external circumferential member to the needle shield remover. The circumferential member comprises protrusions on the external surface configured to engage with engagement members of the cap. When assembling the cap and the needle shield remover, the cap and the circumferential member engage with the engagement members. When an axial and/or rotational movement is applied to the cap, a predefined axial displacement of the cap with respect to the needle shield is available as the circumferential member being engaged with the cap slides along the needle shield removal until it engages with a gripping or engagement member of the needle shield. Thus, the sliding movement of the circumferential member with respect to the needle shield remover stops and any further movement applied to the cap acts on the needle shield remover.

Coupling the removal sequence of the cap or covering from the medicament delivery device to the activation of the supplemental device can be accomplished by connecting the activation member to the cap or other covering located on the medicament delivery device.

Removal of the protective cap from the proximal end of the housing of the medicament delivery device can be accomplished by a pure linear or circumferential motion of the cap relative to housing or by a pure rotation of the cap or a combination of both rotation and axial motions. Regardless of the manner in which the cap is separated from the proximal end of the housing, the activation mechanism of the present disclosure is configured so that the activation member that is attached to the cap will remain securely attached to the cap during cap removal. This will ensure that the normally closed switch can move back to a default position, or the electrical contacts established by removing the insulating material, and thus result in closing the electrical circuit so that the supplemental device is energized by and receives power from the battery.

The protective cap or cover portion of the medicament delivery device is configured and adapted to cover the dose delivery outlet e.g. when mounted on the medicament delivery device or when fixed to the medicament container. In the case where the medicament delivery device is a pen-type injector, it is preferable if the cap has a generally oblong cavity that is configured to receive the oblong portion of the proximal portion of the injector. In some circumstances it may be necessary or desirable to allow a small amount of relative axial movement between the cap and the terminal end of the proximal portion of the medicament delivery device housing.

One possible embodiment of the present disclosure is directed to a system for activating a supplemental device attached to a medicament delivery device, where the medicament delivery device includes a housing having a proximal end, a medicament container positioned within the housing, and a dose delivery outlet accessible through a terminal end of the proximal end. The dose delivery outlet is surrounded by a needle shield acting as a sterile barrier and is attached to a needle shield remover which is mechanically coupled to a removable cap. The supplemental device comprises a battery module that is attached to the housing, preferably to the proximal end of the housing, further comprising an activation member and a communication module wherein the cap movement acts on the activation member to activate the supplemental device. The supplemental device is configured to determine if the cap has moved relative to the needle shield. The communication module is configured to transmit data to an external device where the data comprises the information directly related to the status of the activation member and/or the movement of the removable cap.

In one embodiment the cap is coupled to the needle shield remover and is configured to be movable in an axial and/or circumferential direction, wherein an initial motion sequence of the cap activates the supplemental device but does not move the needle shield remover.

The cap is often assembled with a needle shield remover, or closure member, of a medicament delivery device and is engaged to a needle shield, which protects a needle by hermetically enclosing it. The needle shield may comprise a flexible rubber member in which the needle is embedded, i.e. a flexible needle shield (FNS). For some applications, the FNS is provided with a rigid outer shell, i.e. a rigid needle shield (RNS). To be able to remove the needle shield before use of the medicament delivery device, the needle shield remover is provided with gripping members that engage the needle shield. Since the needle shield remover is attached to the cap, removal of the cap will also pull away the needle shield from the needle, due to the gripping members' engagement with the needle shield. The removable cap is attached to the proximal end of the housing such that the dose delivery outlet is not accessible unless the cap and the needle shield assembly are fully removed from the device.

In one example a continued moving sequence of the cap subsequently to the initial moving sequence of the cap is required to remove of needle shield from the dose delivery outlet.

The activation member is either a switch or an insulating pad.

In one embodiment the battery module or the communication module comprises one or more sensors configured to detect a motion of at least one of the components of the medicament delivery device.

One possible embodiment further comprises a sensor module connectable to any other module and wherein said sensor module comprises one or more sensors configured to detect a motion of at least one of the components of the medicament delivery device.

In one embodiment the switch is initially in a first state (A) where the communication module is prevented from receiving power from the battery and where the movement of the cap unit relative to a proximal end of the medicament delivery device changes the switch to a second state (B) where the battery provides power to the communication module.

Additionally, the communication module is connectable to the battery module or included as part of the battery module and configured to transmit data to an external device. A recorder can be included as part of the battery module or as part of the communication module and the recorder is configured to obtain and store information about the medicament delivery device. The switch preferably is initially in a first state where the recorder is prevented from/does not receive power from the battery and where movement of the cap proximally relative to the housing changes the switch to a second state where the battery provides power to the recorder and/or the communication module

In one possible embodiment the activation member is configured to activate the supplemental device only once, and a potential breach of the sterile barrier of the medicament delivery device is indicated when the switch is in the second state (B) or the insulating pad has been removed.

The switch can be configured such that it can be in the first state only once. This is best accomplished by configuring the switch such that it is prevented from/cannot transform from the second state back to the first state. An alternative configuration that will prevent such a transformation involves the use of a normally closed switch that is physically held in a temporary open position or by the use of a sheet, ribbon, or strip of material operatively connected between the battery and the contact pad.

The communication module or the battery module or supplemental device comprises a recorder configured to obtain and store data information about the medicament delivery device.

The data transmission by the communication module starts when the recorder receives power from the battery.

In some cases where a normally closed mechanical switch is used, and when the cap is pulled away from the medicament delivery device housing that it is attached to, moving the supplemental device that is attached to the housing towards the proximal end of the housing, creates a gap between the cap and the terminal edge of the housing. The switch moving with the cap can slide along a portion of the housing it is in contact with such that, upon reaching the terminal edge of the housing, the switch can protrude into the gap between the housing and the cap. It is contemplated that the supplemental device of this disclosure can be provided by itself, i.e., it need not be provided with an assembly including a medicament delivery device and a cover portion or cap. In other words, it can be provided as a standalone unit.

The activation system of the present disclosure is formed when two steps are completed. One step involves the attachment of the supplemental device to the cap of the medicament delivery device and the other step is the removal of the protective cap or other covering that must be removed before the medicament delivery device is ultimately used for its intended design purpose.

In another embodiment, the supplemental device may comprise two or more modules, either integrated or separated and attachable to each other, wherein any of the modules is removable from the housing and is reusable.

For example, a first battery module can be configured to be permanently attached, i.e., non-releasably coupled, to the housing of the cap of the medicament delivery device. An adhesive, a weld, or a one-way snap fit are ways to achieve this permanent attachment. A component module such as the communication module, a sensor module, a log module, etc., may contain other electrical components, such as the recorder, etc. In one example, the component module may be configured to be removably attached to the battery module such that it can then be reused and attached to another battery module. In such a case, the first battery module is disposed of with the housing or the cap of the first medicament delivery device and the removed component module is then attached to a second battery module containing a fresh or new battery. This can occur prior to or after the second battery module is attached to a second medicament delivery device housing or cap. One end of the insulating material would then be fixed to a protective cap or other covering attached to the housing of a second medicament delivery device.

Positioning of the supplemental device onto the cap can be performed by the manufacturer of the medicament delivery device, by a healthcare provider or by a user of the device. The shape of the supplemental device preferably matches or conforms to the shape of the cap of the medicament delivery device where it is attached.

The transfer of information from the supplemental device is prevented to/cannot occur until the supplemental device has been activated by connecting the battery to the electrical circuit containing the other electrical components. Once activated, the transfer of information to the supplemental device concerning the state of the medicament delivery device may be by galvanic or non-galvanic means, e.g. audible, optical, vibration or electromagnetic.

The medicament expelling mechanism may be mechanical or substantially mechanical, i.e., electro-mechanical, it can also include some electronic components. The supplemental device may comprise means for transferring data to an external device, e.g. by wireless IR, RF or optical means but not limited to those.

The supplemental device may further comprise a sensor e.g. a sound sensor that can detect, for example, a rotational sound of the dose setting mechanism, e.g. such as a dose drum. A processing unit can also be arranged for processing the output signal from an optical sensor or other sensors contained in the supplemental device. The sound sensor can be adapted to activate the processing unit when the dosage setting starts manually by rotating a knob. It might also comprise a memory (e.g. RAM random access memory or another kind of memory), integrated into the processing unit or as a separate unit. The memory unit is arranged for receiving and recording the data from the one or more sensors located in the supplemental device. The sound sensor detects the sound of the dosage setting mechanism movement or e.g. a rotational sound of the dose setting drum might be chosen from one of a microphone, an accelerometer and a vibration sensor.

The supplemental device may further comprise a communication module for communicating and transmitting the dosage data to an external device, where the external device can be one of a mobile device such as a mobile or cell phone, a computer and a remote server such as a cloud for recording, storage and monitoring of the injection or inhalation medicament delivery data such as a dosage, time of delivery, date, frequency, medicament, etc. A memory unit can also be located in the supplemental device. The supplemental device might further comprise a resetting button which enables a manual reset of the supplemental device. Such a reset feature is beneficial if the supplemental device or part of the supplemental device is to be reused, for example, on another medicament delivery device.

According to another aspect of the present disclosure, there is presented a method of collecting and recording medicament delivery data, for example, dosage information, time of delivery, frequency, medicament, date, etc. from a medicament delivery device. The medicament delivery device can be a pen-type injector, an auto-injector or an inhaler that has a dosage setting mechanism with a dose indicator, e.g. such as a dose setting drum or the like. If the dose indicator has dosage value indications on its surface and/or a dosage displaying window or opening on the medicament delivery device circumference surface for displaying the dosage values, then the method could include steps of detecting a sound from a dose indicator movement, e.g. a rotational sound from the dose setting drum or another kind of setting mechanism by a sound sensor, and activating a processing unit by the sound sensor.

The supplemental device may contain a control module and a communication module, wherein either one of those modules comprises one or more sensors configured to detect a motion of at least one of the components of the medicament delivery device. It is also feasible that the supplemental device comprises a sensor module connectable to any other module and which comprises one or more sensors configured to detect a motion of at least one of the components of the medicament delivery device. The sensor module may also be included as part of any other module e.g. the battery module or the communication module. Likewise, the supplemental device may further comprise a logging module connectable to any other module or included as part of any other module e.g. the battery module, the communication module, or the sensor module. The logging module is configured to track user behavior of the medicament delivery device, which user behavior is based on a motion detected by the one or more sensors. By tracking the motion of the medicament delivery device, tracking of user behavior for study, training or compliance is improved, which facilitates medicament delivery.

As used in this disclosure, the one or more sensors may generally refer to any kind of sensor capable of detecting movement. Preferably, the one or more sensors may be one or more of the following sensors: a magnetometer, a gyroscope, and/or an accelerometer, wherein high precision tracking of motion is improved.

The system further comprises a memory module connectable to any other module or included as part of any other module. The memory module is configured to store the data and the communication module is configured to transmit the data wirelessly to the external device. Preferably, the memory module is configured to store data from the logging module, wherein tracking is facilitated for a user thereof. The memory module may comprise a non-volatile memory. According to one embodiment, the communication module may be configured to transmit data (either wirelessly or by wire) from the recorder, logging module, or memory module and is capable of real time visualization if the tracking is achieved. The battery module is energized when the cap or other covering on the medicament delivery device that is attached to a proximal end of the medicament delivery device is moved relative to the proximal end.

The medicament delivery device may be an actual delivery device for injection of a medicament or may be a mock-up demo device for human factors study or for training.

According to another aspect, the system of the present disclosure can be a medicament delivery device coupled to the supplemental device and further includes a computer device that is separate from the medicament delivery device and supplemental device, for example a mobile phone having a display device. The communication module could then be configured to transmit wirelessly and/or by wire real time data from the logging module to the computer device separate from the medicament delivery device and/or to a remote location for later analysis. The communication module may also be configured to transmit wirelessly and/or by wire data stored in a memory module from the logging module to the computer device separate from the medicament delivery device or to a remote location for later analysis.

For the above reasons it may be desirable for the supplemental device to include a recorder component as part of or in addition to a sensor component that collects data relating to relative axial cap movement. Stated differently, activation of the supplemental device could then be correlated directly to cap movement or complete removal. In such a design, the activated supplemental device can be programed to send a signal through its communication component to an external device providing notice that relative cap movement has occurred. This notification could be programed to be automatic immediately upon activation of the supplemental device.

Manipulation of the length of the activation member and/or the placement of the supplemental device on the proximal end of housing can allow for activation to occur based on a predetermined cap separation from the terminal end of the housing. A control component in the supplemental device can be adapted to detect such a predetermined event pattern and to provide notification to an external device that the event has occurred. The control component can also create a time log representing the detected event as a function of time.

The battery that can be used in the activation system of the present disclosure is preferably a button battery, sometimes referred to as a coin or watch battery. Such a battery can be single use and disposable or can be rechargeable. Preferably, the battery should have a shelf or non-use life of at least several years, most preferably at least 4 years. Further, once activated, the battery should be able to supply power that yields a run time of approximately 30 days and no shorter than 3 weeks. A battery cover can be provided that allows user access to the battery while the supplemental device is attached to the medicament delivery device housing.

The system of the present disclosure can communicate through a communication component or module in the supplemental device with an external device, for example, a computer or handheld personal digital assistant (PDA), such as a smart phone, tablet, or the like. The data transmission by the communication module can start immediately after the communication module or the recorder receives power from the battery. This data transmission to the external device can occur wirelessly or through a wired connection. In the above system transmission of data between a medicament delivery device and the data collecting device may take place by wireless means, e.g. RF, IR, capacitive or inductive, or by electromagnetic radiation in the optical range. Transmission of data may take place automatically when the data collecting device and a medicament delivery device are in the proximity of each other, e.g. within a given range.

The electronic circuitry may be adapted to perform one or more functions selected from the group consisting of the members: generating data representing the size of a dose set by the medicament expelling mechanism, generating data representing the size of a dose expelled by the medicament expelling mechanism, generating and storing a time log for data representing sizes of doses set by the medicament expelling mechanism, generating and storing a time log for data representing sizes of doses expelled by the medicament expelling mechanism, transmitting data to an external receiver, receiving data from an external transmitter, controlling a display adapted to display user-readable information, controlling indication means adapted to indicate when the secondary power source need to be recharged, and controlling control means adapted to prevent a dose to be set or expelled when the secondary power source need to be recharged.

The external or data collecting device may be in the form of one of the following devices: a BGM, a CGM, a medicament delivery device, a mechanically controlled medicament delivery device, an electronically controlled medicament delivery device, a PDA, a mobile phone, a key ring device, a credit card sized device, a medical hub, router, necklace, smart watch, or disposable monitoring units.

In another embodiment of the present disclosure a method of activating a supplemental device is presented where transmitting data containing information relating to a medicament delivery device occurs when a protective a cap or other cover that is attached to a proximal portion of the medicament delivery device is moved axially relative to the proximal portion of the medicament delivery device housing or is completely removed from the medicament delivery device housing. This method involves the removal of an activation member from electrical contacts that are directly contacted to a battery that is part of an electrical circuit of the supplemental device than can contain a switch that is operatively connected to the battery where a part of the switch is directly connected to the cap. The activation member may be an insulating pad. A recorder can be configured to obtain and store information about the medicament delivery device and a communication module can be used to transmit data recorder by the recorder to an external device.

The switch is initially in a first state where the control module is prevented from/does not receive power from the battery and where movement of the cap proximally relative to the housing changes the switch to a second state where the battery provides power to the battery module, and if included therein, to a communication module. The power from the battery energizes the battery module so that it can accept information about the medicament delivery device. In some cases that information or data is recorded in a recorder and then further transmitted to an external device by a communication module. The supplemental device can also have a feedback signal that triggers when the battery module is activated. The feedback can be an audible and/or tactile signal. The electric circuit, the energy source (battery), a switch, a speaker unit and/or a piezoelectric unit, could all be used for generating the feedback signal. Further, according to another aspect of the present disclosure, the supplemental device could be a control unit having a clock function. Another aspect could involve the control unit measuring the duration of a simulated dose delivery and comparing it with a predetermined time value and controlling the feedback signal to indicate to the user the occurrence of a correct simulated dose delivery or a failed simulated dose delivery. To further improve the quality of the feedback given to the user, the electronic circuit may be arranged to not only indicate the start of needle penetration and/or dose delivery but may also indicate to the user the time needed to press the training device against the training injection site.

The supplemental device may also comprise an antenna operably connected to said communication module and to memory storage elements that contains unique identification data relating to the medicament delivery device.

The supplemental device may also comprise an optical module operably connected to said communication module and to memory storage elements that contain unique identification data relating to the medicament delivery device.

Thus, one of the main ideas of the present disclosure is that the supplemental device is inactive before activation. Depending on the type of technology used, inactive may mean that the circuit is not switched on at all until the battery provides power to the circuitry. Although a conventional coin battery has been described, it within the scope of the present disclosure that the power or energy source to activate the supplemental device may be derived from less conventional sources, such as a photovoltaic panel or the like.

According to another favourable solution, the supplemental device may be arranged in the housing of the cap. With this solution the supplemental device can be easily added to the outside surface of an existing medicament delivery device housing. As such, the supplemental device does not have to be built into the medicament delivery device.

Regarding usable technologies, the electronics circuit of the supplemental device may comprise Bluetooth technology, which has a few advantages. A Bluetooth transmitter may communicate with a smart device, which may not have to be at such a close range as e.g. NFC-technologies. Most smart devices nowadays are arranged with Bluetooth communication circuits, which facilitates the transmission of information from the information transmitter to the smart device. A further advantage with Bluetooth is the possibility that an information transmitter of a medicament delivery device provided to a certain user is bonded to a smart device of said certain user. There is thus a tight connection between the medicament delivery devices that a user receives and is to use and their personal smart devices. Thus, information from specific medicament delivery devices are only transmitted to specific smart devices. These and other aspects of, and advantages with, the present disclosure will become apparent from the following detailed description of the present disclosure and from the accompanying drawings.

### BRIEF DESCRIPTION OF DRAWINGS

In the following detailed description of the present disclosure, reference will be made to the accompanying drawings, of which

FIG. 1A is a schematic representation of one possible embodiment of the present disclosure in a side view in an initial state of the activation member, where the housing of the cap and functional connections were not shown for clarity.

FIG. 1B is a schematic representation of one possible embodiment of the present disclosure in a side view in a final state of the activation member, where the housing of the cap and functional connections were not shown for clarity.

FIG. 2A schematically illustrates a configuration of the switch unit and the cap in a final state as shown in FIG. 1B without the cap housing for visibility.

FIG. 2B schematically illustrates a general circuit diagram according to the present disclosure, where the medicament delivery device is not shown for clarity.

FIG. 3A schematically shows the configuration of the activation member and the cap with the switch in an initial state before the movement of the cap where the part of the housing of the cap and the functional connections were not shown for clarity.

FIG. 3B is a partial cross-sectional view of FIG. 3A with the cap unit with a needle shield remover or closure member with the activation member in an initial state before the movement of the cap and the before any movement of the needle shield remover

FIG. 4A schematically shows the configuration of the activation member and the cap with the switch in an activated state after the movement of the cap, and before any movement of the needle shield, where part of the housing of the cap and the functional connections were not shown for clarity.

FIG. 4B is a partial cross-sectional view of FIG. 4A with the cap unit with a needle shield remover, or closure member before the sterile barrier is removed.

FIG. 5A schematically shows the configuration of the activation member and the cap with the switch in an activated state after the movement of the cap and after movement of the needle shield, where part of the housing of the cap and the functional connections were not shown for clarity.

FIG. 5B is a partial cross-sectional view of FIG. 5A with the cap unit with a needle shield remover, or closure member after the sterile barrier has been removed.

FIG. 6A illustrates an exploded view of the cap unit with the supplemental device.

FIG. 6B is a schematic view of an alternative embodiment with the supplemental device mounted on the housing and the activation member being an isolating pad in the initial state of the activation member.

FIG. 7 displays a functional scenario according to the present disclosure where the supplemental device integrates with the user and external devices.

### DETAILED DESCRIPTION

In the following description, the wording smart device will be used. In this context, smart devices may include electronic devices that are provided with processors or processing circuitry that are capable of running computer programs as well as storage space to store programs as well as data retrieved from different external sources. The processing circuitry may for example use any combination of one or more of a suitable central processing unit (CPU), multiprocessor, microcontroller, digital signal processor (DSP), application specific integrated circuit (ASIC), field programmable gate arrays (FPGA) etc., capable of executing any herein disclosed operations concerning detection and or transmission of data. It is further to be understood that the smart devices are provided with communication systems that are capable of communicating with data networks in order to access different databases. It is to be understood that databases may be accessed via the internet, so called cloud services, and/or databases that are connected directly to and accessed via local area networks. It is further to be understood that the smart devices in this context comprise some sort of human-machine interface for two-way communication. The human-machine interface may comprise displays, keyboards, microphones, loudspeakers, I/O-ports for connection of peripherals. Further the smart devices may be provided with antennas for wireless communication with the networks. Also, the smart devices may be arranged with receiving and transmitting mechanisms capable of communicating with NFC tags as well as programs capable of establishing and handling the communication with the NFC tags. Also, the smart devices may be arranged with receiving and transmitting mechanisms capable of communicating via Li-Fi (light-fidelity) technology.

Further, in the following description, the wording medicament delivery device will be used. In this context, medicament delivery devices may include a number of devices capable of delivering certain doses of medicament to a user, such as e.g. injection devices with or without injection needles, inhalers of all kinds, such as powder, aerosol driven, gas, nebulizers having mouth or nasal pieces, dispensers for medicament in tablet form. The medicament delivery devices may be of either disposable type or re-usable type and may be provided with medicament containers suitably arranged for specific medicaments in specific forms.

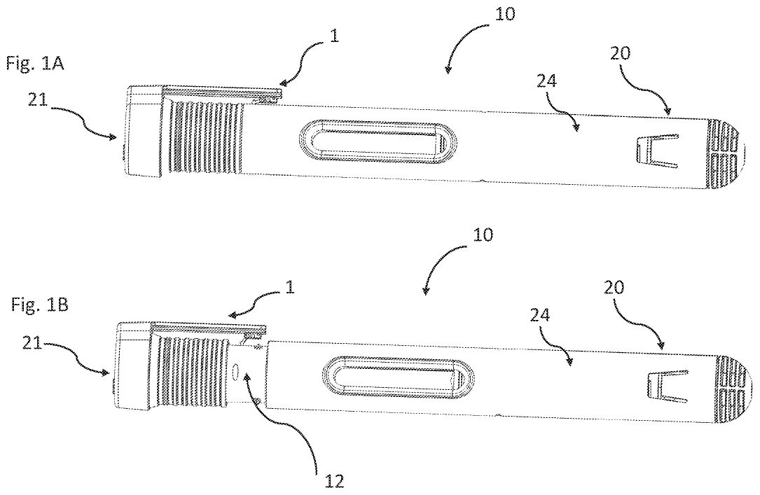

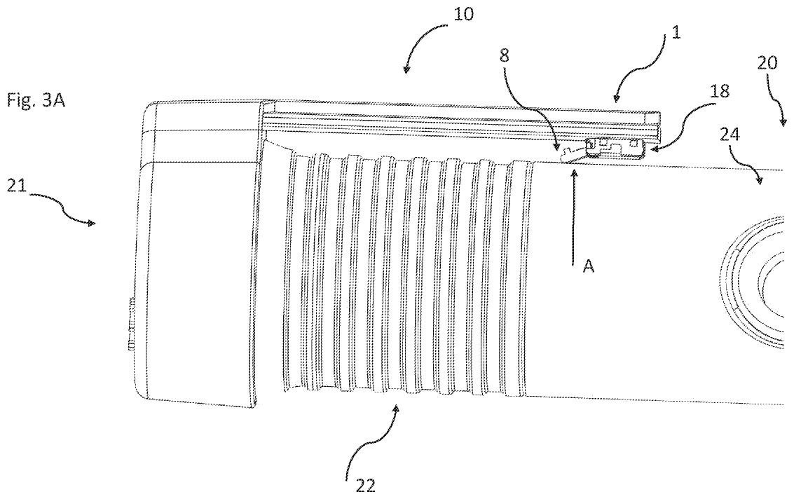

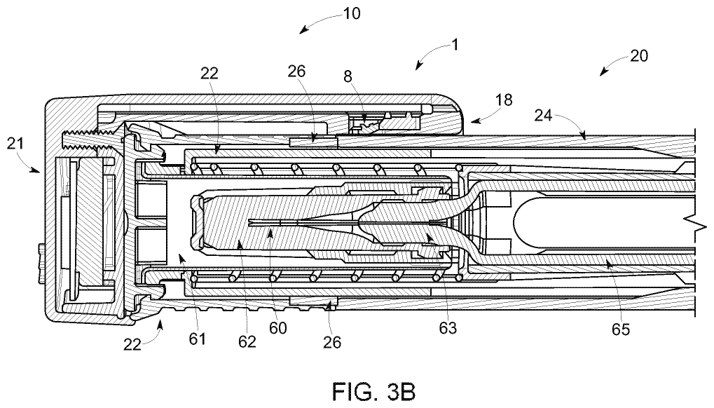

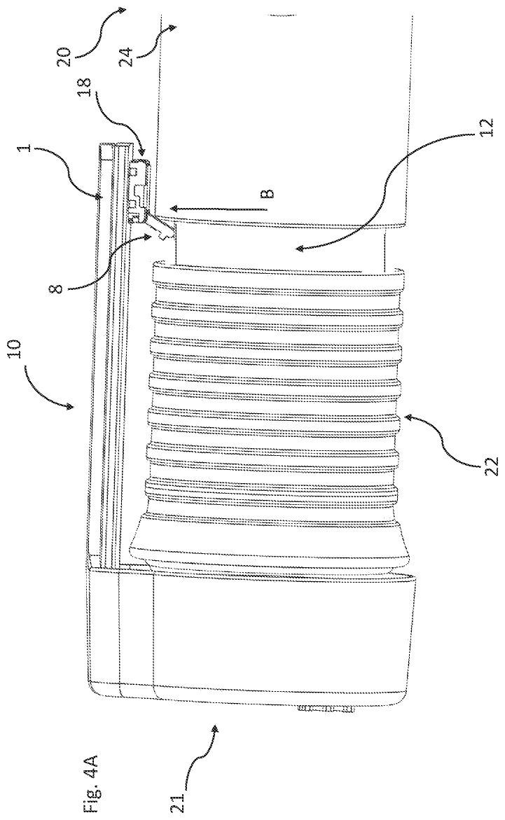

Referring to FIG. 1A and FIG. 1B representing a possible configuration 10 of an activation system for a medical delivery device 20, where the supplemental device 1 is attached to the cap 21 of the medicament delivery device 20 having a housing 24 containing a medicament container 65 having a dose delivery outlet 60 i.e. a fixed needle that is covered by needle shield 62 Attached to protective cap 21 is needle shield remover 61, where the removal of the cap 21 from housing 24 necessarily causes removal of the needle shield 62 from needle as shown in FIGS. 3B, 4B and 5B. FIG. 1A shows a side view of the medicament delivery device with the activation member 8 attached to the supplemental device 1 mounted on the cap 21 where the housing surrounding the activation member 8 is not shown for clarity.

FIG. 1A further represents a configuration 10 when the activation member 8 preferably is initially in a first state where the recorder is prevented from/does not receive power from the battery 2 and where movement of the cap 21 proximally relative to the housing 24 changes the activation member 8 to a second state as shown in FIG. 1B where the battery provides power to the recorder and/or the communication module.

The activation member 8 can be configured such that it can be in the first state only once. This is best accomplished by configuring the activation member 8 such that it is prevented from/cannot transform from the second state back to the first state. One possible configuration that will prevent such a transformation involves the use of normally closed switch that is physically held in a temporary open position.

The activation member 8 is configured to be in contact with the housing 24 of the medicament delivery device 20 when the supplemental device 1 is mounted on the medicament delivery device 20. Stated differently, the activation member 8 is arranged to be slidably in contact with the surface of the proximal end of the medical delivery device 20 as represented in FIG. 1A and FIG. 3A. When the supplemental device 1 is mounted on an existing injection device, such as an auto-injector, that has a protective cap 21 that surrounds and encloses a separate needle cover or shield 62, and a pulling or twisting motion is applied to the cap 21 by a user, the cap will move towards the proximal end of the device 20.

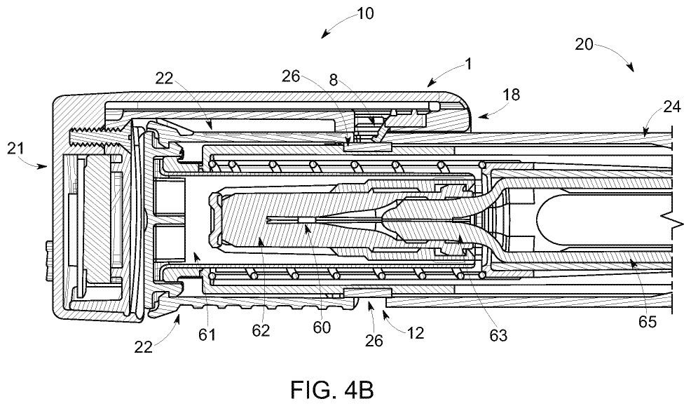

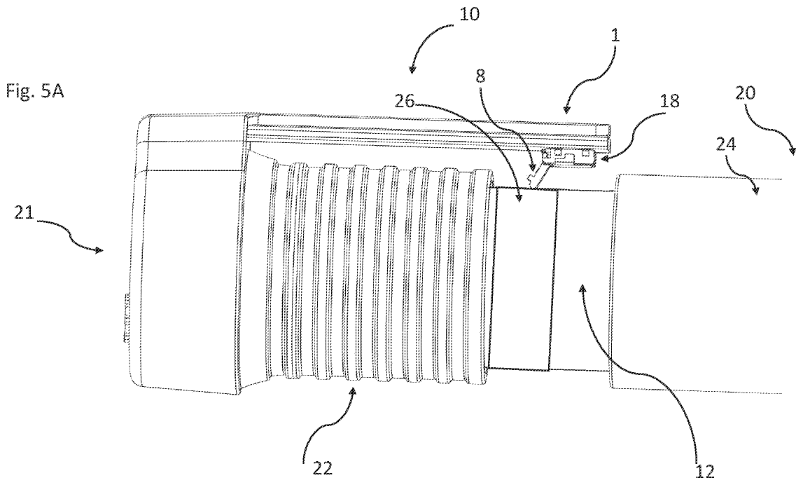

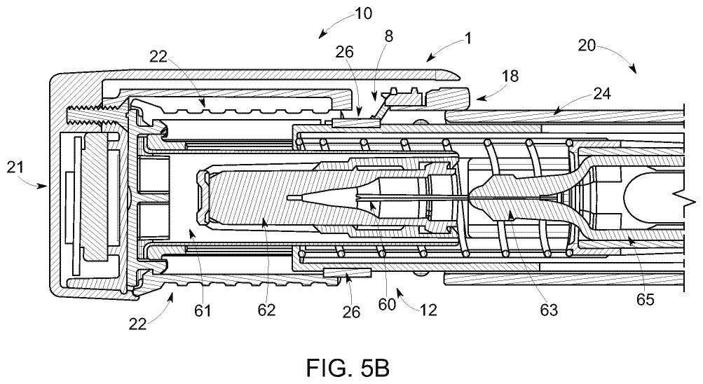

This axial movement of the cap 21 towards the proximal end of the device 20 creates a gap 12 between the housing 24 and the cap 21 as illustrated in FIG. 1B, FIG. 4A and FIG. 5A The cap 21 abuts the proximal end of the housing 24 such that the activation member 8 being a switch in this example, is in mechanical contact with the housing 24 of the medicament delivery device 20 as shown in FIG. 1A and FIG. 3A. Upon movement of the cap 21 toward the proximal end of the delivery device, the switch 8, triggered by the movement of the cap 21, slides along the housing 24 of the medicament delivery device 20. When the switch 8 reaches the edge of the proximal end of the housing 24, the switch can slide vertically into the space 12 created by the distancing of the cap 21 from the housing 24 of the medical delivery device 20 as shown in FIG. 1B, FIG. 4A and FIG. 5A. This vertical movement with respect to the longitudinal axis of the medicament delivery device triggers the switch 8 back to its default position and closes the circuit, to form an electrical connection such that electricity from the battery will flow through and between the contacts, thus closing or energizing the electrical circuit connected to the other electrical components. The normally closed switch 8 may have alternative spatial or functional configurations, collectively referred to as activation member.

In one embodiment the coupling between the protective cap 21 and the needle shield remover 62 may be configured to allow a predefined axial displacement of the cap 12 with respect to the needle shield remover 62, and this axial movement does not act on the needle shield remover 62 to remove the needle shield.

One example may comprise an additional elongation between the gripping members of the cap and the needle shield remover. Stated differently the cap 21 may slide with an axial and/or rotational movement along the needle shield remover 62, defining an axial displacement 12, before gripping members of the needle shield and/or gripping members of the cap 21 can engage with each other. Upon coupling of the gripping members, any further axial and/or rotational movement of the cap 21 translates into a movement of the needle shield remover 62 and the attached rigid or flexible needle shield.

One embodiment may be configured to provide an additional external circumferential member 26 to the needle shield remover 62 or to the internal circumferential side of the cap 21. The circumferential member 26 comprises protrusions on the external or internal surface configured to engage with engagement members of the cap 21 or the needle shield remover 62. When assembling the cap 21 and the needle shield remover 62, the cap and the circumferential member 26 engage with the engagement members. When an axial and/or rotational movement is applied to the cap 21, a predefined axial displacement 12 of the cap 21 with respect to the needle shield is available as the circumferential member being engaged with the cap 21 slides along the needle shield removal until its engages with a gripping or engagement members of the needle shield. Thus, the sliding movement of the circumferential member with respect to the needles shield remover stops and any further movement applied to the cap 21 acts on the needle shield remover 62.

Alternatively, the circumferential member is coupled to the needle shield remover 62 and the cap 21 may slide on the circumferential member 26 until it abuts gripping or engagement members and the sliding movement of the circumferential member with respect to the cap 21 stops and any further movement applied to the cap 21 acts on the needle shield remover 62.

The activation of the supplemental device 1, 3 prior to the removal of the cap 21, 31 and the needle shield 62, may be used to check the status of the medicament delivery device with the external device. For example, information about the content and the administration can be stored and or displayed on the external device. When the supplemental device is activated and the data has been transmitted, wirelessly or via a wired connection, to the external device, the external device may verify the status of the content, i.e. the temperature if the sensors comprise temperature sensors, date of manufacturing, verifying if the content is safe to use, or if patient data is available, the personal administration plan may be displayed.

An advantage amongst others is the additional layer of safety, as sterility breaches or temperature breaches or safe use of the content may be verified and communicated from the external device to the user. For example, a temperature logging of the device over the lifetime can be transmitted wirelessly and the medicament shelf life calculated based on the transmitted data, and the external device may communicate to the user if the medicament is still safe to use and until when. Temperature conditions may in some cases accelerate or slowdown the degradation or contamination of some medicaments, accordingly the shelf life may be reduced or extendable. This might be a non-negligible advantage in regions where the medical delivery devices being stored in ambient environment with non-constant temperatures.

Another possible use of the activation mechanism is to verify before removal of the cap and thus whether there has been a breach of the sterility, if the medication is the intended one and if the administration is the right one. For example a user with visual and/or mental disabilities, who may not be able to distinguish visually or by tactile means between several medicament delivery devices containing different medicaments or medicament doses, or may not remember the personal administration plan, can activate the supplemental device and verify if it is the intended medication before self-administration of the medication.

For example, in a medical environment with a higher number of medicament delivery devices and users, the activation of the supplemental device may be coupled to an external device verifying and monitoring the administration plans for several users, thus minimizing accidental or erroneous administrations of the medication contained in the medicament delivery device.

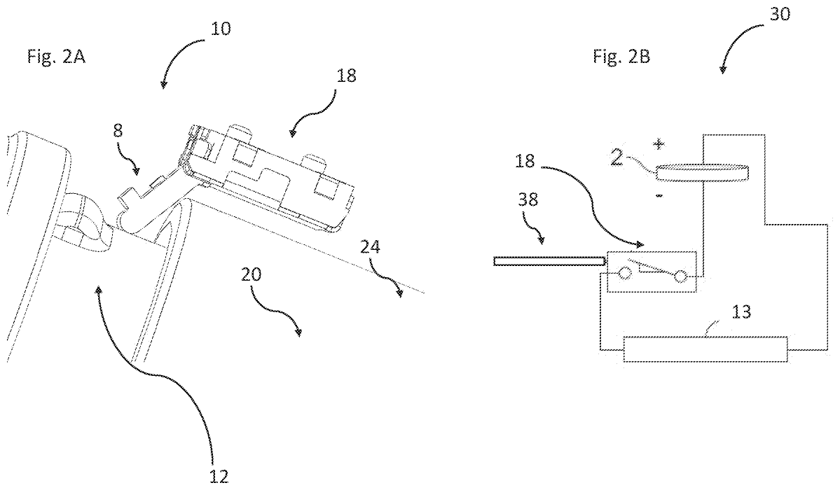

As shown in FIG. 4B the axial displacement of the cap that triggered the activation member 8 in an initial default state A to move into a second state B which establishes a connection to the power supply and thus activates the supplemental device 1 as shown in FIG. 2A and FIG. 2B. This initial motion sequence of the cap activates the supplemental device 1 without inducing any movement of the needle shield remover. Stated differently the initial motion sequence activating the supplemental device is not projected as a sequence of motion onto the needle shield remover 62.

Especially in the case of a medicament delivery device that relies upon sterility of the dose delivery outlet, very little cap movement is tolerated before the supplemental device is activated. As illustrated in FIG. 4B relatively little cap movement, which translates into an axial displacement of the cap unit towards the proximal end of the medical device is required to activate the supplemental device 1 without triggering any needle shield remover 62 displacement or movement. Any additional displacement or movement of the cap beyond the motion sequence required for activating the supplemental device may trigger the needle shield remover 62. This is especially the case when the dose delivery outlet is an injection needle having its own separate flexible needle shield that is axially fixed to the protective cap. In other words, additional axial movement of the cap away from the device housing can result in axial movement of the needle shield away from the proximal end of the needle which can destroy the condition of sterility as illustrated in FIG. 5B.

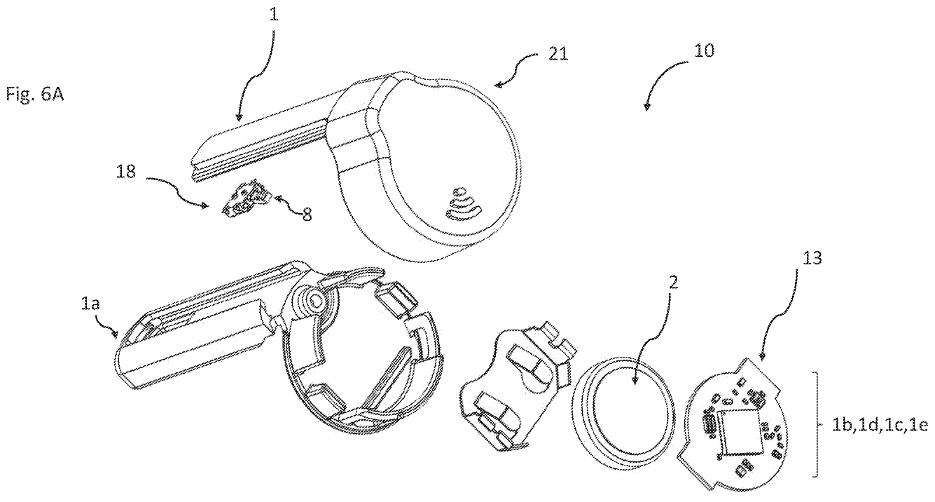

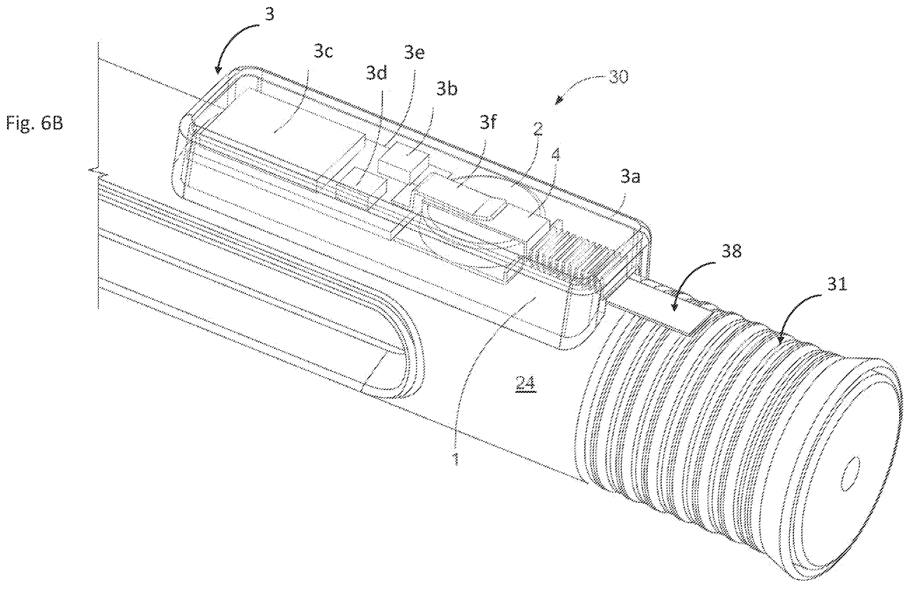

FIG. 6A shows an embodiment including a number of electrical components, for instance a communication module 13 comprising a communication unit 1b, a recorder 1d having data storage, and a processor or control unit 1c. All these components/modules are in electrical communication through circuit board 1e and enclosed by cover 1a. Further a battery module 18 comprising the activation member being communicatively coupled to an energy source shown as a coin battery 2 is represented and the communication module 13 not shown in the Figures.

The communication unit 1b contained in the supplemental device 1 of the present disclosure may also make use of radio frequency identification technology, RFID. In particular, high frequency RFID provides a number of advantages regarding communication. The possibilities of using HF RFID are numerous and in particular provides the use of Near Field Communication, NFC. NFC is particularly suitable because it is a set of standards for smartphones and other smart devices to establish radio communication. NFC is a set of short-range wireless technologies, typically requiring a distance of 10 cm or less. NFC operates at 13.56 MHz on ISO/IEC 18000-3 air interface and at rates ranging from 106 kbit/s to 424 kbit/s. NFC always involves an initiator and a target; the initiator actively generates an RF field that can power a passive target. This enables NFC targets to take very simple form factors such as tags, stickers, key fobs, or cards that do not require batteries.

In the following description of the technology used the word NFC-tag may be used. In this context it is to be understood that an NFC-tag will comprise an NFC-chip connected to a circuit as well as an antenna. An NFC-tag is not limited to being integrated in a patch or label, but may be a stand-alone unit, or integrated in the material used for manufacturing medicament delivery devices. Further, the NFC-tag may include further features and components that are needed for the required or desired purposes and applications as will be apparent below.

NFC tags contain data and are typically read-only but may be rewriteable. They can be custom encoded by their manufacturers or use the specifications provided by the NFC Forum, an industry association charged with promoting the technology and setting key standards. The tags can securely store personal data such as debit and credit card information, loyalty program data, PINs and networking contacts, among other information.

Regarding medicament delivery devices, they can be arranged with NFC tags in order to perform a number of tasks. The NFC tags may be arranged as part of the above-mentioned supplemental devices of the present disclosure and conveniently incorporated in labels which are attached to an outer surface of a housing of a medicament delivery device.

The types of short-range wireless technology (SRWT) that may be utilized in the supplemental devices include ANT+, RFID, Zigbee and Bluetooth. One favourable technology is Bluetooth technology. Bluetooth technology operates in the unlicensed industrial, scientific and medical (ISM) band at 2.4 to 2.485 GHz, using a spread spectrum, frequency hopping, full-duplex signal at a nominal rate of 1600 hops/sec. The 2.4 GHz ISM band is available and unlicensed in most countries. Especially the Bluetooth low energy or Bluetooth Smart may be utilized in connection with medicament delivery devices and the functions that are requested. The Bluetooth circuit is provided with a transmitter that is capable of transmitting for instance a unique identification number or data as well as recording a time stamp. The Bluetooth circuit is preferably driven by the power source described above, e.g. the coin battery, and the Bluetooth circuit for activating the Bluetooth circuit only when there is an occurrence or change of status of the medicament delivery device.

Alternatively, Li-Fi technology which is based on light produced by LEDs instead of Radio frequency spectrum produced by Wi-Fi may be used in circumstances where Wi-Fi or other radiation may cause interference or disturb other equipment in specific areas like hospitals. The communication module 1b contained in the supplemental device 1 of the present disclosure may also make use of a photodetectors and LED light bulbs.

Turning to FIG. 2B, the basic circuit diagram of the supplemental device 1 that can be used in the present activation system is schematically shown. An energy source 2, shown as a coin battery, is electrically connected to a control module 1c, which may be connected or incorporated to the communication module 13. This communication module may contain one or more sensors/sensor module as described above, a communication unit 1b for transmitting data to an external device, a recorder or memory module, and a controller 1c (control unit). The sensors may include a magnetometer, a gyroscope, and/or an accelerometer. A magnetometer may be used to detect the position of an auto injector needle of the medicament delivery device with the supplemental device attached to it. A gyroscope may be used to detect the position of the medicament delivery device and the accelerometer may be used to detect movement in a particular direction. By use of a combination of a gyroscope and three accelerometers, a high precision tracking may be provided. The supplemental device of FIG. 2B also contains an energy isolation feature comprising a normally closed mechanical switch mechanism that is mechanically held open until the cap is removed which then causes the switch to close and completes the circuit.

The memory module, when present, can be a non-volatile memory to store data from the sensor(s)/sensor module and/or from a logging module. The non-volatile memory may e.g. be a read-only memory, flash memory, ferroelectric RAM (F-RAM), most types of magnetic computer storage devices (e.g. hard disks, floppy disks, and magnetic tape), optical discs. The non-volatile memory may be removable, to be inserted into a reader of an external device. The supplemental device may further be provided with a contact interface, allowing stored data to be uploaded to an external device through a cable connection. The communication module when present allows real data and/or stored data to be uploaded to the external device. The communication module is configured to transmit (wirelessly and/or by wire) real time data from the logging module or to transmit stored data from the memory module. The communication module may be provided with e.g. wi-fi, Bluetooth, BLE or Li-Fi.

Removal of cap 21 from housing 24 causes a movement with respect to the longitudinal axis of the medicament delivery device which triggers the switch 8 back to its default position, which represents the translation of the activation member. This will then close the circuit allowing the battery 2 to activate and energize the electrical components such as a communication module 1b, the recorder 1d having data storage, and the processor or control unit 1c.

In another possible embodiment of the presently disclosed activation system, the design of the supplemental device is such that it can be removed from the medical delivery device 20 and reused.

FIG. 6B illustrates an alternative activation system and embodiment representing a possible configuration 30 of an activation system for a medical delivery device 20, where the supplemental device 3 is attached to the cap 31 of the medicament delivery device 20 having a housing 24 containing a medicament container 65 having a dose delivery outlet 60 i.e. a fixed needle that is covered by needle shield 62. Attached to protective cap 31 is needle shield remover 61, where the removal of the cap 31 from housing 24 necessarily causes removal of the needle shield 62 from the needle. FIG. 6B shows a side view of the medicament delivery device 20 with the activation member 38 attached to the supplemental device 3 mounted on the housing 24 of the medicament delivery device 20 and coupled to the cap 31.

FIG. 6B further shows the supplemental device 3 including several electrical components for instance a communication module 3b, a recorder 3d having data storage, and a processor or control unit 3c. All these components/modules are in electrical communication through circuit board 3e and enclosed by cover 3a. Further a battery module shown as a coin battery 2. The battery module can include a contact pad 38 and a switch 48 operatively connected to the battery 2 and the contact pad 38, where a part of the switch 48 is directly connected to the removable cap 31 when the battery module is attached to the housing 24 or where a part of the switch is directly connected to the housing when the battery module is attached to the cap 31.

In another embodiment of the present disclosure a method of activating a supplemental device 3 is presented where transmitting data containing information relating to a medicament delivery device occurs when a protective a cap 31 or other cover that is attached to a proximal portion of the medicament delivery device 20 is moved axially relative to the proximal portion of the medicament delivery device housing or is completely removed from the medicament delivery device housing. This method involves the removal of an activation member, e.g., an insulating sheet or strip, from electrical contacts that are directly contacted to a battery that is part of an electrical circuit of the supplemental device 3 that can contain a switch 48 that is operatively connected to the battery 2 and to a contact pad 38, where a part of the switch 48 is directly connected to the cap 31. A recorder can be configured to obtain and store information about the medicament delivery device 20 and a communication module can be used to transmit data recorder by the recorder to an external device.

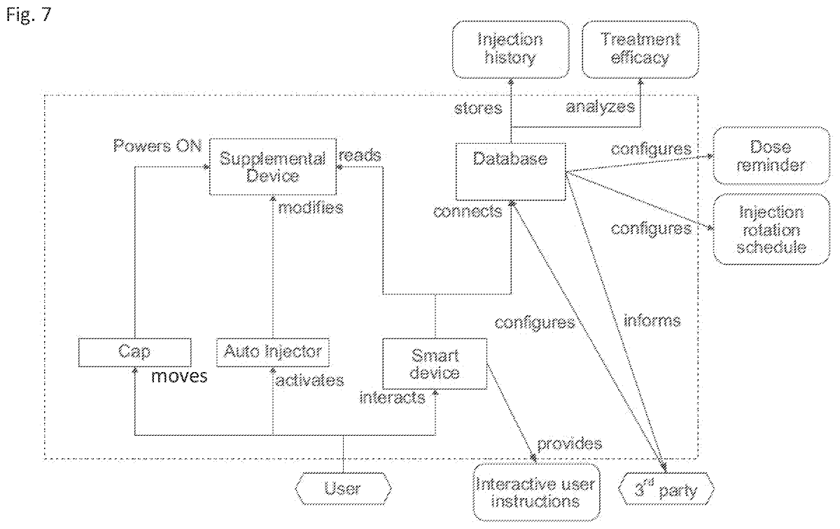

FIG. 7 illustrates one possible logical flow diagram representative of activation system of the present disclosure. Circuits may be connected to a number of modules or components within the supplemental device for providing status information. Such status information may comprise end of dose delivery. It may for example be important for a user to know when an injection sequence has ended and that it is safe to remove the device from the injection site. In this case a circuit may be affected by moving components at the end of dose delivery, wherein the circuit acts as a switch. The switch information detected by the SRWT is transmitted to the smart device, wherein the smart device is arranged to indicate to the user that the device may safely be removed. Also, this information confirms that the device is used.

The circuits and switches may further be used as interactive, step by step, instructions. For example, the smart device may be provided with an instruction application showing a user in a stepwise manner how a device should be handled. When one step has been performed, whereby a certain circuit has been affected and detected by the SRWT and transmitted to the smart device, an OK or positive response is provided by the smart device and displayed to the user. The instruction application then shows the subsequent handling step to be taken. In this manner, all steps affect different circuits that in turn provide the SRWT-chip with status information. This status information is successively transmitted to the smart device and appropriate information is displayed to the user by the instruction application.

In connection with the increased integration of the medicament delivery device and the smart device by the supplemental device, further information could be collected in order to increase the understanding of the effects of a certain treatment scheme, e.g. disease monitoring. The programs or applications that are used in the smart device in connection with the medicament delivery devices may further include questionnaires that are filled in by the user in connection with a dose delivery operation. The questionnaire may include a number of questions regarding the current status of the patient and may preferably be configurable depending on therapy, disease and user needs. The areas that might be handled may include quality of life, cognitive function, pain, fatigue, nausea, mental health, etc. The answers of the questionnaire may then be transmitted from the smart device to external databases together with information collected via the SWRT-tags for processing and evaluation to find positive or negative correlations between the treatment scheme and type of medicament in relation to the perceived condition of the patient.

Further, if the smart device is not equipped with an NFC-reader, the attachment could be provided with such an NFC-reader, thereby adding functionality to the smart device. The integration of the medicament delivery device and the smart device further provides real time interactive user instructions as well as correct injection times, dates and dose quantities because of the close connection between the medicament delivery device and the smart device because of real time reading of the NFC-tag. The injection times, dates and dose quantities can be recorded directly in the smart device for further processing or transmittal.

Many smart devices are arranged with motion sensors in three dimensions, which functionality could be used in connection with handling of the medicament delivery device. For instance, the smart device could detect how it, and thus the medicament delivery device, is being held. This may be important for some types of medicaments and for some types of medicament delivery devices in that the medicament delivery device has to be held in a certain way during some steps when used. This could for example be a medicament delivery device using a so-called dual chamber medicament container, where it can be important how the medicament container is held during mixing and priming. The motion sensors of the smart device could then be used to detect how the medicament delivery device is held and could inform a user on how to hold the device and alert the user if the device is not held according to instructions.

Further features of the smart device that could be used with the integrated medicament delivery device include the use of a camera that is often an integrated part of the smart device. The camera is used to receive the light fidelity or alternatively could then be used to take photographs of the content of the medicament container, which often is transparent, in order to obtain information regarding the status of the medicament. For example, colouring or opacity of a medicament may indicate that something adverse has happened to the medicament, such as exposure to temperatures outside the prescribed range, such that the medicament should not be used. The comparison of colour or opacity may be performed directly by the user in an application in the smart device, or the picture may be sent by the smart device to an external site where skilled personnel perform the comparison and alert the patient of any deficiencies of the medicament and advice as to how proceed.

Regarding adherence and patient responsibility, there are features and functions of the smart device that may be utilized. Some medicaments and treatment schemes are very expensive to the national healthcare authorities and a lot of responsibility is put on the users to really adhere to the treatment schemes. There has been discussions in several countries in the world that if patients do not adhere to an expensive treatment, they should be forced to pay for the continued treatment, fully or partly, the arguments being that those persons that are not interested enough in a treatment should have to pay for it. The information and medicament delivery history obtained from the NFC-tags could be used to monitor the adherence.

In that respect, biometrical sensors such as fingerprint sensors, eye and/or face recognition via cameras on the smart devices mat provide proof of a user of a certain medicament delivery device, providing proof that it is the legitimate user that has activated the medicament delivery device for delivering a dose. Biometrical sensors may further be used in order to ascertain that the device is prevented to/cannot be accidentally, or wilfully, used by a third person.

For example, if a larger battery is used, the NFC-tag could use the temperature sensor that is built into the NFC-chip. This may be an advantage because then the temperature of a medicament delivery device and/or a medicament container may be monitored and logged for instance during transport. This might be important for a number of medicaments that are temperature sensitive, whereby it can be ensured that the quality of the medicament has not been affected by temperature variations outside approved ranges. Also, the temperature sensor could be used to provide information when a medicament has reached a target temperature for delivery. The information is then communicated to the smart device, where the latter provides handling and temperature information to the user.

It is to be understood that the embodiments described above and shown in the drawings are to be regarded only as non-limiting examples of the present disclosure and that it may be modified in many ways within the scope of the patent protection.

1-14: (canceled)

15: A medicament delivery system comprising:
a drug delivery device comprising a medicament container positioned within a housing, a dose delivery outlet accessible through a terminal end of the housing and a needle shield acting as a sterile barrier, coupled to a needle shield remover;
a supplemental device comprising a battery module, an activation member and a communication module;
a movable cap coupled to the needle shield remover wherein a cap movement acts on the activation member to activate the supplemental device;
wherein the supplemental device is configured to determine if the cap has moved relative to the needle shield and wherein the communication module is configured to transmit data to an external device where the data comprises information directly related to the status of the activation member and/or the movement of the removable cap.

16: The medicament delivery system of claim 15, wherein the cap when coupled to the needle shield remover is configured to be movable in an axial and/or circumferential direction and wherein an initial motion sequence of the cap activates the supplemental device but does not move the needle shield remover.

17: The medicament delivery system of claim 15, wherein the dose delivery outlet is prevented from being accessible unless the cap is fully removed from the medicament delivery device, wherein a continued moving sequence of the cap subsequently to the initial moving sequence of the cap is required to remove of needle shield from the dose delivery outlet.

18: The medicament delivery system of claim 15, wherein the battery module or the communication module comprises one or more sensors configured to detect a motion of at least one of the components of the medicament delivery device.

19: The medicament delivery system of claim 15, wherein the system further comprises a sensor module connectable to any other module and wherein said sensor module comprises one or more sensors configured to detect a motion of at least one of the components of the medicament delivery device.

20: The medicament delivery system of claim 15, wherein the activation member is one of a switch or an insulating pad.

21: The medicament delivery system of claim 20, wherein the switch is initially in a first state where the communication module is prevented to receive power from the battery and where movement of the cap unit relative to a proximal end of the medicament delivery device changes the switch to a second state where the battery provides power to the communication module.

22: The medicament delivery system of claim 20, wherein the activation member is configured to activate the supplemental device only once, and wherein the switch being in the second state or the insulating pad being removed, indicates a potential breach of the sterile barrier of the medicament delivery device.

23: The medicament delivery system of claim 15, wherein the communication module or the battery module or supplemental device comprises a recorder (1d,3d) configured to obtain and store data information about the medicament delivery device.

24: The medicament delivery system of claim 15, wherein the data transmission by the communication module starts when the recorder receives power from the battery.

25: The medicament delivery system of claim 15, wherein any of the modules is removable from the housing and is reusable.

26: The medicament delivery system of claim 15, wherein the system further comprises a logging module connectable to any other module or included as part of any other module, and wherein the logging module is configured to start tracking motion of the medicament delivery device when the cap unit is removed.

27: The medicament delivery system of claim 15, wherein the system further comprises a memory module connectable to any other module or included as part of any other module, and wherein the memory module is configured to store the data and the communication module is configured to transmit the data wirelessly to the external device.

28: The medicament delivery system of claim 15, wherein the communication module comprises means for wireless data transfer and signaling means for the status of the activation member, and or the movement of the cap.

29: A medicament delivery system comprising:
a drug delivery device having a medicament container positioned within a housing, a dose delivery outlet accessible through a terminal end of the housing and a needle shield surrounding the dose delivery outlet to provide a sterile barrier, where the needle shield is operatively associated with a needle shield remover;
a supplemental device comprising a battery module, an activation member and a communication module; and
a removable cap is operatively associated with the needle shield remover, where an initial movement of the cap causes the activation member to activate the supplemental device and where subsequent movement of the cap causes the needle shield to be removed from the dose delivery outlet,
wherein the supplemental device is configured to determine if the cap has moved relative to the needle shield, and
wherein the communication module is configured to transmit data to an external device where the data comprises information directly related to the status of the activation member or the movement of the removable cap.

30: The medicament delivery system of claim 29 further comprising a sensor module containing a sensor that detects a motion of the cap relative to the housing.

31: The medicament delivery system of claim 29, wherein the activation member comprises a switch or an insulating pad.

32: The medicament delivery system of claim 29, wherein the activation member comprises a switch that is initially in a first state that prevents a battery from energizing the communication module and where movement of the cap relative to the terminal end of the housing moves the switch to a second state where the battery provides power to the communication module.

33: The medicament delivery system of claim 29, wherein the cap can move axially relative to the needle shield remover a first distance that will activate the activation member, where further axial movement of the cap with cause axial movement of the needle shield remover which in turn will cause axial movement of the needle shield relative to the dose delivery outlet.

34: The medicament delivery system of claim 29, wherein the supplemental device is removable from the drug delivery device and is attachable to and reusable with a second drug delivery device.
